(19) United States
(12) Patent Application Publication
(10) Pub. No.: US 2020/0284781 A1
(43) Pub. Date: Sep. 10, 2020

(54) TEST METHOD OF COMBINED TOXICITY FOR CHLORPYRIFOS AND BUTACHLOR

(71) Applicant: ZHEJIANG ACADEMY OF AGRICULTURAL SCIENCES, ZHEJIANG (CN)

(72) Inventors: YANHUA WANG, ZHEJIANG (CN); FEIDI WANG, ZHEJIANG (CN); XINQUAN WANG, ZHEJIANG (CN); XINJU LIU, ZHEJIANG (CN); GUILING YANG, ZHEJIANG (CN); XUEHUA AN, ZHEJIANG (CN); QIANG WANG, ZHEJIANG (CN)

(73) Assignee: ZHEJIANG ACADEMY OF AGRICULTURAL SCIENCES

(21) Appl. No.: 16/806,035

(22) Filed: Mar. 2, 2020

(30) Foreign Application Priority Data
Mar. 6, 2019 (CN) 201910169549.6

(51) Int. Cl. G01N 33/50

(52) U.S. Cl. CPC G01N 33/5014

(57) ABSTRACT

A test method for the combined toxicity of chlorpyrifos and butachlor, which comprises the following steps: A. Experimental organisms: The zebrafish wild type AB strain is used in the experiment. After the purchase, it is domesticated in the laboratory, and the experimental fishes for collecting fish eggs have been kept in this laboratory for more than 1 month. Through the combined toxicity test and single pesticide test, it is convenient to increase the reference data and improve the mutual comparison of the data according to the impact of different environments and different agents on the animals. Better balance and offset the effects of irrelevant variables, making the experimental results more convincing. The probit analysis method is used to calculate the pesticides on larvae based on the number and the time of death of fish larvae, and the data is reasonably analyzed and processed to avoid reliance on single effect.

### TECHNICAL FIELD

The invention belongs to the technical field of toxicity testing, and particularly relates to a method for testing the combined toxicity of chlorpyrifos and butachlor.

### BACKGROUND

With the rapid development of modern industry and agriculture, more and more organic pollutants enter the environment through various routes and coexist in the environment. For a long time, the research of toxicology has focused on the single chemical toxicity effects research. Many standards, such as the allowable discharge standards for wastewater and safe concentration standards, are also established based on the toxic effects of single chemical substances. At present, many environmental toxicological effects cannot be explained by the action mechanism of a single pollutant, A number of studies have shown that even if multiple pollutants are at the “safe” level of the relevant water quality benchmarks, which may have a significant combined toxic effect on aquatic organisms. Relevant evaluation, standards based on single-effect pollution cannot truly reflect reality Therefore, in order to accurately and effectively assess the environmental risks of pollutants in environmental water, and to formulate environmental thresholds that are “safer” for aquatic organisms, the combined toxicity of pollutants needs to be considered.

There have been many studies on the single toxic effects of chlorpyrifos and butachlor on aquatic organisms, but their combined toxicity to aquatic organisms has not been reported. As a model organism, zebrafish is often used in toxicology tests because of its high homology with the human genome. Zebrafish embryos are optically transparent and one can clearly observe various stages of development, helping to observe the pathological changes in the body and the toxic effects in the development of somites. In addition, zebrafish have the advantages of low breeding costs and high reproduction rates. Japanese medaka has been recognized by most world organizations as a model experimental animal and was listed by the international Organization for Standardization as one of the toxicological test species in the 1980s. The study of single and combined toxic effects also provides a scientific basis for monitoring the pollution of the farmland environment. The existing experimental methods are complicated and the experimental results are unclear, which cannot provide basic data for the study of combined pollution.

### SUMMARY

The purpose of the present invention is to provide a test method for the combined toxicity of chlorpyrifos and butachlor in order to solve the above problems, and solve the shortcomings of the existing equipment.

In order to solve the above problems, the present invention provides a technical solution:

The test method for the combined toxicity of chlorpyrifos and butachlor, comprising the following steps:

A. Experimental organisms: Using a zebrafish wild-type AB strain in the experiment and domesticated in laboratory after purchase, the fishes for collecting experimental fishes eggs had been raised in this laboratory for more than a month and fed with the fairy shrimps twice a day, remove the bait and feces 30 minutes after feeding; a ratio of light time to dark time is 14 h:10 h, on the eve of breeding, putting the healthy and sexually mature brood stock into the mating spawning tank with a ratio of female to male 1:2, 8 h earlier the next day light to fertilize their eggs, separating the cleaned and disinfected normal fertilized eggs into two parts: using one part for embryo experiments; incubating the other part in a 26±1° C. light incubator, using the larvae for exposure experiment after swimming balance; breeding Japanese medaka in a 10-liter round glass tank with a female-to-male ratio of 3:2 per 50 species of fish, and the breeding water is 8 L, freshly hatched larvae are fed twice daily in the morning and evening; every morning after collecting the fertilized eggs from the female, separating the eggs with a dropper, and selecting the fertilized healthy fertilized eggs for hatching larvae;

B. Experimental water and experimental equipment: The preparation method of experimental water refers to the OECD guidelines, and it will be used after being fully exposed to oxygen, its main indicators are: water temperature of zebrafish is 26±1 and Japanese medaka 25±1° C., pH value is 7.8±0.2, dissolved oxygen 7.8 mg·L⁻¹, hardness recorded as 230±20 mg·L⁻¹ respectively, as embryo and larvae poisoning equipment;

C. Experimental reagents: 96% chlorpyrifos technical product and 95% butachlor technical product, using analytical pure N, N-dimethylformamide and Tween-80 to dissolve the pesticide technical product and make it into a certain concentration of stock solution, its additive volume ratio is not more than 0.1% for determination;

D. Toxicity of pesticides to zebrafish embryos: On the basis of clearing the effective concentration range of pesticides in pre-tests, diluting the pesticide stock solution with standard dilution water to 5-7 concentrations with a geometrical ratio, and using a 24 well cell culture dish for exposure apparatus, the volume of each well is 3 mL, 20 wells are the same experimental concentration, and the remaining 4 wells are blank controls; during the experiment, of test solution and 1 randomly selected 3 hpf (hourpost-fertilization) normal fertilized embryo at the embryo shield stage were put into every well, set up 3 replicates at each concentration, every culture dish as a replicate, and incubating in a multifunctional incubator at 26±1° C. with a photoperiod of 14 h (light): 10 h (dark);

E. Single toxicity of pesticides to zebrafish larvae and Japanese medaka: Design the toxicity test of pesticides to zebrafish and Japanese medaka according to the method of the OECD guidelines, based on the preliminary test to determine the effective concentration range of the pesticide, diluting the stock solution with standard dilution water to 5-7 concentrations in equal steps, using a 24-well cell culture dish as the poisoning device, the volume of each well is 3 mL, adding 2 mL of test solution to each well and a larva that has developed normally and just entered the migratory period through microscopy during the experiment, no feeding during the test, each concentration is set up in triplicate, the zebrafish test temperature is 26±1° C., the test temperature is 25±1° C., the photoperiod is 14 h (light): 10 h (dark), replacing the test solution every 24 h, observing and counting the number of dead larvae every 24 h, and calculating LC₅₀ values and their 95% confidence limits by the probit analysis method when exposed 24 h, 48 h, 72 h and 96 h;

F. Combined toxicity test: The toxicity test is performed on zebrafish larvae and Japanese medaka larvae, the test procedure is as follows:

a. Zebrafish larvae: The LC₅₀ value of zebrafish larvae with a single pesticide for 96 h is a toxic unit, and 5-7 different concentrations with a geometrical ratio, test method and calculation of LC₅₀ value of each exposure time are the same as 1.4.2;

b. Japanese medaka: A single pesticide is used to measure the LC₅₀ value of Japanese medaka for 96 hours, mixing chlorpyrifos and butachlor to form binary mixed systems with different ratios of 1:4, 2:3, 1:1, 3:2 and 4:1, according to the pre-experiment results, 5-7 different concentrations are set at equal logarithmic intervals to determine the combined toxicity of the mixed system to Japanese medaka, the method is the same as the determination of single toxicity, the total concentration of the binary mixture is the sum of the concentrations of the two components;

G. Combined toxicity evaluation method: Using the following formula to find the sum of biological toxicity S: S=Am+Ai+Bm/Bi, wherein Am and Bin are the toxicities of each poison in the mixture, and Ai and Bi are the toxicities of A and B poisons when acting alone; convert S into additive index AI, when S≤1, AI=(1/S)−1.0; when S>1, AI=1.0−S, and evaluating the compound effect of chemicals with AI, when −0.2<AI<0.25, that is, addition; when it is AI≥0.25, it is greater than the addition effect, that is, synergistic effect; when AI≤−0.2 is less than the additive effect, that is, antagonism;

H. Data processing: Calculating the LC₅₀ value of pesticides on larvae and their 95% confidence limits by probit analysis based on the number and time of dead fish larvae, and using 95% confidence limit of LC₅₀ as the criterion to determine whether the toxicity difference of different pesticides is significant, LC₅₀≤0.1 mg a.i. L⁻¹, is hypertoxic; 0.1<LC₅₀≤1.0 mg a.i. L⁻¹, is high toxicity; 1.0<LC₅₀≤10.11 mg a.i. L⁻¹, is medium toxicity; LC₅₀>10.0 mg a.i. L⁻¹ is low toxicity. The maximum allowable concentration of MPC employs 100 as the protection factor, the formula is: MPC=96 h-LC₅₀/100, to get the maximum allowable concentration of a poison.

Preferably, the test water temperature in step A is controlled at 25±1° C. and the light cycle is 14 h of light and 10 h of darkness.

Preferably, in step B, using a Lycra S8AP0 type apochromatic stereo microscope for observation and photographing.

Preferably, the larvae in the migratory period in step E refer to fish 120 h after fertilization of eggs.

Preferably, the mixing ratio in the step F is designed with reference to a more toxic agent.

Preferably, the MPC in the step I is the maximum allowable concentration.

Preferably, in step I, the toxicity classification standard of pesticides for larvae is based on “Environmental Safety Evaluation Test Guidelines of Chemical Pesticides” formulated by the State Environmental Protection Administration in 1989.

Preferably, in the step D, the test solution needs to be replaced every 24 h, observing and microscopical observing the CK group and the exposed group every 24 h, recording the number of embryos with normal development and malformations, and calculating the number of embryos hatched and larvae malformations, the experiment lasts 96 hours.

Beneficial effects of the present invention: The method is efficient and accurate. By combining a combined toxicity test and a single pesticide test, it is convenient to increase the reference data and improve the mutual comparison of data according to the effects of different environments and different agents on animals. The effects of irrelevant variables are offset, making the experimental results more convincing Probit analysis is used to calculate pesticides on larvae based on the number and the time of dead fish larvae, and the data is reasonably analyzed and processed to avoid relying on a single effect. Relevant evaluation standards of pollution are convenient to reflect the objective requirements of the actual environmental quality, improve the environmental risk of effective assessment of pollutants in environmental water, and facilitate the formulation of environmental thresholds that are “safer” for aquatic organisms.

### DESCRIPTION OF THE EMBODIMENTS

This specific embodiment adopts the following technical scheme: The test method for the combined toxicity of chlorpyrifos and butachlor includes the following steps:

A. Experimental organisms: Using a zebrafish wild-type AB strain in the experiment and domesticated in laboratory after purchase, the fishes for collecting experimental fishes eggs had been raised in this laboratory for more than a month and fed with the fairy shrimps twice a day, remove the bait and feces 30 minutes after feeding; a ratio of light time to dark time is 14 h:10 h, on the eve of breeding, putting the healthy and sexually mature brood stock into the mating spawning tank with a ratio of female to male 1:2, 8 h earlier the next day light to fertilize their eggs, separating the cleaned and disinfected normal fertilized eggs into two parts: using one part for embryo experiments; incubating the other part w in a 26±1° C. light incubator, using the larvae for exposure experiment after swimming balance; breeding Japanese medaka in a 10-liter round glass tank with a female-to-male ratio of 3:2 per 50 species of fish, and the breeding water is 8 L, freshly, hatched larvae are fed twice daily in the morning and evening; every morning after collecting the fertilized eggs from the female, separating the eggs with a dropper, and selecting the fertilized healthy fertilized eggs for hatching larvae;

B. Experimental water and experimental equipment: The preparation method of experimental water refers to the OECD guidelines, and it will be used after being fully exposed to oxygen, its main indicators are: water temperature of zebrafish is 26±1° C., and Japanese medaka 25±1° C., pH value is 7.8±0.2, dissolved oxygen ≥7.8 mg·L⁻¹, hardness recorded as 230±20 mg·L⁻¹, respectively, as embryo and larvae poisoning equipment;

C. Experimental reagents: 96% chlorpyrifos technical product and 95% butachlor technical product, using analytical pure N, N-dimethylformamide and Tween-80 to dissolve the pesticide technical product and make it into a certain concentration of stock solution, its additive volume ratio is not more than 0.1% for determination;

D. Toxicity of pesticides to zebrafish embryos: On the basis of clearing the effective concentration range of pesticides in pre-tests, diluting the pesticide stock solution with standard dilution water to 5-7 concentrations with a geometrical ratio, and using a 24-well cell culture dish for poisoning apparatus, the volume of each well is 3 mL, 20 wells are the same experimental concentration, and the remaining 4 wells are blank controls; during the experiment, 2 mL of test solution and 1 randomly selected 3 hpf (hourpost-fertilization) normal fertilized embryo at the embryo shield stage were put into every well, set up 3 replicates at each concentration, every culture dish as a replicate, and incubating in a multifunctional incubator at 26±1° C. V: with a photoperiod of 14 h (light): 10 h (dark);

E. Single toxicity of pesticides to zebrafish larvae and Japanese medaka: Design the toxicity test of pesticides to zebrafish and Japanese medaka according to the method of the OECD guidelines, based on the preliminary test to determine the effective concentration range of the pesticide, diluting the stock solution with standard dilution water to 5-7 concentrations with a geometrical ratio, using a 24-well cell culture dish as the poisoning device, the volume of each well is 3 mL, adding 2 mL of test solution to each well and a larva that has developed normally and just entered the migratory period through microscopy during the experiment, no feeding during the test, each concentration is set up in triplicate, the zebrafish test temperature is 2.6±1° C., the Japanese medaka test temperature is 25±1° C., the photoperiod is 14 h (light): 10 h (dark), replacing the test solution every 24 h, observing and counting the number of dead larvae every 24 h, and calculating LC₅₀ values and their 95% confidence limits by the probit analysis method when exposed 24 h, 48 h, 72 h and 96 h;

F. Combined toxicity test: The toxicity test is performed on zebrafish larvae and Japanese medaka larvae, the test procedure is as follows:

a. Zebrafish larvae: The LC₅₀ value of zebrafish larvae with a single pesticide for 96 h is a toxic unit, and 5-7 different concentrations with a geometrical ratio, test method and calculation of LC₅₀ value of each exposure time are the same as 1.4.2;

b. Japanese medaka: A single pesticide is used to measure the LC₅₀ value of Japanese medaka for 96 hours, mixing chlorpyrifos and butachlor to form binary mixed systems with different ratios of 1:4, 2:3, 1:1, 3:2 and 4:1, according to the pre-experiment results, 5-7 different concentrations with a geometrical ratio are set at equal logarithmic intervals to determine the combined toxicity of the mixed system to Japanese medaka the method is the same as the determination, of single toxicity, the total concentration of the binary mixture is the sum of the concentrations of the two components;

G. Combined toxicity evaluation method: Using the following formula to find the sum of biological toxicity S: S=Am/Ai+Bm/Bi, wherein Am and Urn are the toxicities of each pesticide in the mixture, and Ai and Bi are the toxicities of A and B pesticides when acting alone; convert S into additive index AI, when S≤1, AI=(1/S)−1.0; when S>1, AI=1.0−S, and evaluating the compound effect of chemicals with AI, when −0.2<AI<0.25, that is, addition; when it is AI≥0.25, it is greater than the addition effect, that is, synergistic effect; when AI≤−0.2 is less than the additive effect, that is, antagonism;

H. Data processing: Calculating the LC₅₀ value of pesticides on larvae and their 95% confidence limits by probit analysis based on the number and time of dead fish larvae, and using 95% confidence limit of LC₅₀ as the criterion to determine whether the toxicity difference of different drugs is significant, LC₅₀≤0.1 mg a.i. L⁻¹, is hypertoxic; 0.1<LC₅₀≤1.0 mg a.i. L⁻¹, is high toxicity; 1.0<LC₅₀≤10.0 mg a.i. L⁻¹, is medium toxicity; LC₅₀>10.0 mg a.i. L⁻¹, is low toxicity, the maximum allowable concentration of MPC employs 100 as the protection factor, the formula is: MPC=96 h-LC₅₀/100, to get the maximum allowable concentration of a poison.

Wherein the test water temperature in step A is controlled at 25±1° C., and the light cycle is 14 h of light and 10 h of darkness, for the survival of experimental organisms.

Wherein in step B, using a Lycra S8 AP0 type apochromatic stereo microscope for observation and photographing, for observing the changes of experimental organisms.

Wherein the larvae in the migratory period in step E refer to fish 120 h after fertilization of eggs, for improving the accuracy of the test.

Wherein the mixing ratio in the step F is designed with reference to a more toxic agent, saving test time.

Wherein the MPC in the step I is the maximum allowable concentration, for analyzing data.

Wherein in step I, the toxicity classification standard of pesticides for larvae is based on “Environmental Safety Evaluation Test Guidelines of Chemical Pesticides” formulated by the State Environmental Protection Administration in 1989, providing effective reference for data analysis.

Wherein in the step D, the test solution needs to be replaced every 24 h, observing and microscopical observing the CK group and the exposed group every 24 h, recording the number of embryos with normal development and malformations, and calculating the number of embryos hatched and larvae malformations, the experiment lasts 96 hours, for structural analysis.

### EXAMPLES

(1) Toxicity of Chlorpyrifos and Butachlor to Zebrafish Embryos:

After 96 hours of exposure, the mortality of zebrafish embryos in both the blank control group and the adjuvant control group was <10%. The LC₅₀ value of chlorpyrifos on zebrafish embryos at 24 hours was 170.1 (84.25-401.7) mg a.i. L⁻¹. The toxicity of chlorpyrifos increases with the prolonged exposure time. When exposed to 96 h, the toxicity increases significantly. Its LC₅₀ value is 13.03 (7.5449.71) mg a.i. L⁻¹. The LC₅₀ value of butachlor on zebrafish embryo at 24 h was 32.79 (23.26-63.39) mg al, L⁻¹. The toxicity increased significantly with the prolonged exposure time. The LC₅₀ values at 48 h, 72 h and 96 h were 5.82 (4.33-9.02) and 4.42 (3.04-6.42) and 1.93 (1.37-3.55) mg a.i. L⁻¹, butachlor is 6.75 times more toxic to zebrafish embryos than chlorpyrifos at 96 h.

TABLE 1
Single toxicity of chlorpyrifos and butachlor to zebrafish embryos

| | Exposure time | | LC₅₀ |
|---|---|---|---|
| Pollutant | (h) | Slope | (95% CI) mg a.i. L⁻¹ |
| Chlorpyrifos | 24 | 2.89 | 170.1 (84.25-401.7) |
| | 48 | 2.24 | 119.7 (68.08-554.6) |
| | 72 | 2.15 | 63.41 (41.37-129.3) |
| | 96 | 2.18 | 13.03 (7.54-19.71) |
| Butachlor | 24 | 3.38 | 32.79 (23.26-63.39) |
| | 48 | 3.92 | 5.82 (4.33-9.02) |
| | 72 | 2.95 | 4.42 (3.04-6.42) |
| | 96 | 3.29 | 1.93 (1.37-3.55) |

Chlorpyrifos and butachlor exposure have effects on multiple phylogeny of zebrafish embryos, mainly manifested as egg coagulation, pericardial edema, yolk sac edema, and spinal curvature, as shown in Table 1;

(2) Single Toxicity of Chlorpyrifos and Butachlor to Zebrafish Larvae and Japanese Medaka Larvae:

After 96 hours of exposure, the mortality rates of zebrafish larvae and Japanese medaka larvae were <10% in the blank control group and the adjuvant control group. The LC₅₀ value of chlorpyrifos ors zebrafish larvae was 0.67 (0.54-1.06) mg a.i. L⁻¹ at 24 hours of exposure. With the increase of exposure time, the toxicity of chlorpyrifos to zebrafish larvae increased. The LC₅₀ value of 96 h exposure was 0.27 (0.12-0.38) rug a.i. L⁻¹, The LC₅₀ value of butachlor cars zebrafish larvae for 24 h was 0.67 (0.534.06) mg a.i. L⁻¹. With the increase of exposure time, the toxicity of butachlor to zebrafish larvae increased, but the difference was not significant. The LC₅₀ value after exposure for 96 hours was 0.44 (0.30-0.58) mg a.i. L⁻¹. Because the 95% confidence limits of the LC₅₀ value of chlorpyrifos and butachlor on zebrafish larvae at 96 h. overlap, there is no significant difference between the two toxicity to zebrafish larvae at 96 h, as shown in Table 2;

The LC₅₀ value of chlorpyrifos to Japanese medaka larvae at 24 h was 0.75 (0.56-1.13) mg a.i. L⁻¹. With the increase of exposure time, the toxicity of chlorpyrifos to Japanese medaka larvae increased, but the difference was not significant. The LC₅₀ value after exposure for 96 h was 0.24 (0.06-0.38) mg a.i. L⁻¹. The LC₅₀ value of butachlor on Japanese medaka larvae at 24 h was 0.85 (0.56-1.46) mg a.i. L⁻¹. As the exposure time increased, the toxicity of butachlor ran Japanese medaka larvae increased, but the difference was not significant. The LC₅₀ value after exposure for 96 h is 0.43 (0.18-0.62) mg a.i. L⁻¹. Because the 95% confidence limits of the LC₅₀ values of chlorpyrifos and butachlor on Japanese medaka larvae at 96 h overlap, there is no significant difference in the toxicity between them to zebrafish larvae at 96 h, as shown in Table 3.

TABLE 2
Single toxicity of chlorpyrifos and butachlor to zebrafish larvae

| | Exposure time | | LC₅₀ |
|---|---|---|---|
| Pollutant | (h) | Slope | (95% CI) mg a.i. L⁻¹ |
| Chlorpyrifos | 24 | 3.73 | 0.67 (0.54-1.06) |
| | 48 | 5.39 | 0.39 (0.23-0.50) |
| | 72 | 5.38 | 0.34 (0.18-0.44) |
| | 96 | 4.43 | 0.27 (0.12-0.38) |
| Butachlor | 24 | 3.73 | 0.67 (0.53-1.06) |
| | 48 | 3.96 | 0.59 (0.43-0.86) |
| | 72 | 3.88 | 0.51 (0.66-0.71) |
| | 96 | 4.76 | 0.44 (0.30-0.58) |

TABLE 3
Single toxicity of chlorpyrifos and
butachlor to Japanese medaka larvae

| | Exposure time | | LC₅₀ |
|---|---|---|---|
| Pollutant | (h) | Slope | (95% CI) mg a.i. L⁻¹ |
| Chlorpyrifos | 24 | 4.06 | 0.75 (0.56-1.13) |
| | 48 | 3.33 | 0.40 (0.22-0.56) |
| | 72 | 3.44 | 0.35 (0.17-0.49) |
| | 96 | 3.74 | 0.24 (0.06-0.38) |
| Butachlor | 24 | 2.67 | 0.85 (0.56-1.46) |
| | 48 | 3.41 | 0.72 (0.19-1.04) |
| | 72 | 3.27 | 0.54 (0.30-0.76) |
| | 96 | 3.18 | 0.43 (0.18-0.62) |

The toxicity of chlorpyrifos and butachlor to zebrafish larvae and Japanese medaka larvae was not significantly different, and both were toxic in high toxicity grades; the maximum allowable concentrations of chlorpyrifos and butachlor to zebrafish larvae are 0.0027 mg a.i. L⁻¹ and 0.0044 mg a.i. L⁻¹, respectively; the maximum allowable concentrations of the above two pesticides to Japanese medaka larvae are 0.0024 mg a.i. L⁻¹ and 0.0043 mg a.i. L⁻¹, respectively.

(3) Combined Toxicity of Chlorpyrifos and Butachlor to Zebrafish Larvae and Japanese Medaka Larvae:

The 96-hour LC₅₀ value obtained from the single toxicity of chlorpyrifos and butachlor to zebrafish larvae was a toxicity unit, and a 1:1 combined toxicity test was performed. The results showed that under the 1:1 ratio of toxicity of the two pesticides, the combined effect was mainly antagonistic. With 24 h to 72 h exposure it is the antagonistic effect and with 96 h exposure it is the additive effect. The toxicity of butachlor was reduced by the presence of toxic chlorpyrifos, and the toxicity of chlorpyrifos was also reduced by the presence of butachlor. The antagonism weakened with the prolonged exposure time, and it showed an additive effect after exposure to 96 h. Therefore, the longer the organism is in contact with it, the greater the threat it may be. As shown in Table 4, when chlorpyrifos and butachlor are formulated at a concentration ratio of 1:1, they will be harmful to zebrafish within 24 to 96 hours. The combined toxicity of larvae is shown in Table 4;

TABLE 4
Combined toxicity of chlorpyrifos and butachlor to zebrafish larvae

| | | LC₅₀ | | | |
|---|---|---|---|---|---|
| | Exposure | (95% CI) mg a.i. L⁻¹ | | Additive | |
| Proportion | time (h) | chlorpyrifos | butachlor | index AI | Action |
| Equitoxic ratio ratio | 24 | 0.55 (0.39-1.74) | 0.91 (0.65-2.90) | −1.15 | Antagonism |
| | 48 | 0.43 (0.32-0.82) | 0.72 (0.54-1.37) | −1.32 | Antagonism |
| | 72 | 0.27 (0.19-0.58) | 0.45 (0.31-0.97) | −0.68 | Antagonism |
| | 96 | 0.16 (0.11-0.24) | 0.26 (0.18-0.41) | −0.15 | Antagonism |
| Equivalent | 24 | 0.18 (0.13-0.40) | 0.18 (0.13-0.40) | 0.79 | Synergism |
| concentration | 48 | 0.16 (0.11-0.31) | 0.16 (0.11-0.31) | 0.47 | Synergism |
| | 72 | 0.14 (0.10-0.25) | 0.14 (0.10-0.25) | 0.46 | Synergism |
| | 96 | 0.11 (0.076-0.33) | 0.11 (0.076-0.33) | 0.57 | Synergism |

Chlorpyrifos and Butachlor were antagonistic to Japanese medaka in five concentration ratios (1:4, 2, 3:3, 1:1, 3:2, and 4:1) after exposure from 24 h to 96 h. At a concentration ratio of 2:3, the antagonism weakened with the extension of the exposure time, and at a concentration ratio of 1:1, the antagonism increased with the exposure time, as shown in Tables 5-9;

TABLE 5
Combined toxicity of chlorpyrifos and butachlor
at a ratio of 1:4 to Japanese medaka

| Exposure | LC₅₀ (95% CI) | | Additive | |
|---|---|---|---|---|
| time | mg a.i. L⁻¹ | | index | |
| (h) | chlorpyrifos | butachlor | AI | Action |
| 24 | 0.57 | 2.29 | −2.44 | Antagonism |
| | (0.42-1.01) | (1.69-4.03) | | |
| 48 | 0.33 | 1.30 | −1.61 | Antagonism |
| | (0.23-0.63) | (0.94-2.52) | | |
| 72 | 0.28 | 1.14 | −4.76 | Antagonism |
| | (0.21-0.50) | (0.84-2.01) | | |
| 96 | 0.22 | 0.88 | −1.93 | Antagonism |
| | (0.16-0.33) | (0.65-1.32) | | |

TABLE 6
Combined toxicity of chlorpyrifos and butachlor
at a ratio of 2:3 to Japanese medaka

| Exposure | LC₅₀ (95% CI) | | Additive | |
|---|---|---|---|---|
| time | mg a.i. L⁻¹ | | index | |
| (h) | chlorpyrifos | butachlor | AI | Action |
| 24 | 1.52 | 2.29 | −3.69 | Antagonism |
| | (1.04-3.39) | (1.57-5.08) | | |
| 48 | 0.44 | 0.66 | −1.01 | Antagonism |
| | (0.33-0.66) | (0.49-0.99) | | |
| 72 | 0.28 | 0.43 | −0.58 | Antagonism |
| | (0.20-0.50) | (0.31-0.75) | | |
| 96 | 0.23 | 0.35 | −0.73 | Antagonism |
| | (0.17-0.36) | (0.26-0.54) | | |

TABLE 7
Combined toxicity of chlorpyrifos and butachlor
at a ratio of 1:1 to Japanese medaka larvae

| Exposure | LC₅₀ (95%) CI | | Additive | |
|---|---|---|---|---|
| time | mg a.i. L⁻¹ | | index | |
| (h) | chlorpyrifos | butachlor | AI | Action |
| 24 | 1.53 | 1.53 | −2.81 | Antagonism |
| | (1.04-3.44) | (1.04-3.44) | | |
| 48 | 1.22 | 1.22 | −3.72 | Antagonism |
| | (0.89-2.25) | (0.89-2.25) | | |
| 72 | 0.93 | 0.93 | −3.35 | Antagonism |
| | (0.69-1.44) | (0.69-1.44) | | |
| 96 | 0.81 | 0.81 | −4.12 | Antagonism |
| | (0.56-2.11) | (0.56-2.11) | | |

TABLE 8
Combined toxicity of chlorpyrifos and butachlor
at a ratio of 3:2 to Japanese medaka

| Exposure | LC₅₀ (95% CI) | | Additive | |
|---|---|---|---|---|
| time | mg a.i. L⁻¹ | | index | |
| (h) | chlorpyrifos | butachlor | AI | Action |
| 24 | 2.23 | 1.48 | −3.68 | Antagonism |
| | (1.56-4.84) | (1.04-3.23) | | |
| 48 | 1.94 | 1.29 | −5.62 | Antagonism |
| | (1.41-3.80) | (0.94-2.53) | | |
| 72 | 1.31 | 0.87 | −4.33 | Antagonism |
| | (0.98-1.98) | (0.65-1.32) | | |
| 96 | 0.99 | 0.66 | −4.49 | Antagonism |
| | (0.73-2.22) | (0.49-1.48) | | |

TABLE 9
Combined toxicity of chlorpyrifos and butachlor at a
ratio of 4:1 concentration to Japanese medaka larvae

| Exposure | LC₅₀ (95% CI) | | Additive | |
|---|---|---|---|---|
| time | mg a.i. L⁻¹ | | index | |
| (h) | chlorpyrifos | butachlor | AI | Action |
| 24 | 0.80 | 0.20 | −0.29 | Antagonism |
| | (0.57-1.19) | (0.14-0.30) | | |
| 48 | 0.61 | 0.15 | −0.73 | Antagonism |
| | (0.45-1.12) | (0.11-0.28) | | |
| 72 | 0.43 | 0.11 | −0.43 | Antagonism |
| | (0.29-1.18) | (0.073-0.29) | | |
| 96 | 0.14 | 0.036 | 0.55 | Antagonism |
| | (0.10-0.25) | (0.0255-0.0625) | | |

The combined action modes of chlorpyrifos and butachlor are different at different concentration ratios, and as time goes by, the change law of the strength of the combined effects at different concentration ratios is also different. It can be seen that the combined effects of the two pesticides are very complicated. This is consistent with the generalized theory of combined effects proposed by Zhou Qixing. They believe that in addition to the physical and chemical properties of pollutants, the relationship of concentration combinations of pollutants plays a more direct and more important role under the conditions of multiple composite pollution. Different organisms species have different reaction modes for each pollutant and the interaction between pollutants under the same compound pollution condition, Naturally, in organisms, sometimes interactions occur not only between pollutants and pollutants, but between pollutants and the inherent components of the organism itself. It is because of the mechanism of existence of the organism, which makes different biological species face the same type of composite pollution stress and produce different ecotoxicological effects, making the same concentration and the same type of pollution stress lead to different biological accumulation. Sometimes, despite being the same biological species, they also have different ecotoxicological effects on the same type of combined pollution stress due to different populations. Therefore, the combined mechanism of chlorpyrifos and butachlor is still unclear, and further research is needed. The basic principles and main features of the present invention and the advantages of the present invention have been shown and described above. Those skilled in the art should understand that the present invention is not limited by the above embodiments. What is described in the above embodiments and description is only illustrative of the present invention. Various modifications and improvements can be made without departing from the principle and scope, of the present invention, these modifications and improvements shall all fall within the scope of the claimed invention, the claimed scope of the invention is defined by the appended claims and their equivalents.

What is claimed is:

1. A test method for the combined toxicity of chlorpyrifos and butachlor, which comprises the following steps:
A. Experimental organisms: Using a zebrafish wild-type AB strain in the experiment and domesticated in laboratory after purchase, the fishes for collecting experimental fishes eggs had been raised in this laboratory for more than a month and fed with the fairy shrimps twice a day, remove the bait and feces 30 minutes after feeding; a ratio of light time to dark time is 14 h:10 h, on the eve of breeding, putting the healthy and sexually mature brood stock into the mating spawning tank with a ratio of female to male 1:2, 8 h earlier the next day light to fertilize their eggs, separating the cleaned and disinfected normal fertilized eggs into two parts: using one part for embryo experiments; incubating the other part in a 26±1° C. light incubator, using the larvae for exposure experiment after swimming balance; breeding Japanese medaka in a 10-liter round glass tank with a female-to-male ratio of 3:2 per 50 species of fish, and the breeding water is 8 L, freshly hatched larvae are fed twice daily in the morning and evening; every morning after collecting the fertilized eggs from the female, separating the eggs with a dropper, and selecting the fertilized healthy fertilized eggs for hatching larvae;
B. Experimental water and experimental equipment: The preparation method of experimental water refers to the OECD guidelines, and it will be used after being fully exposed to oxygen, its main indicators are: water temperature of zebrafish is 26±1° C., and Japanese medaka 25±1° C., pH value is 7.8±0.2, dissolved oxygen 7.8 mg hardness recorded as 230±20 mg·L⁻¹ respectively, as embryo and larvae poisoning equipment;
C. Experimental reagents: 96% chlorpyrifos technical product and 95% butachlor technical product, using analytical pure N, N-dimethylformamide and Tween-80 to dissolve the pesticide technical product and make it into a certain concentration of stock solution, its additive volume ratio is not more than 0.1% for determination;
D. Toxicity of pesticides to zebrafish embryos: On the basis of clearing the effective concentration range of pesticides in pre-tests, diluting the pesticide stock solution with standard dilution water to 5-7 concentrations with a geometrical ratio, and using a 24-well cell culture dish for poisoning apparatus, the volume of each well is 3 mL, 20 wells are the same experimental concentration, and the remaining 4 wells are blank controls; during the experiment, 2 mL of test solution and 1 randomly selected 3 hpf (hourpost-fertilization) normal fertilized embryo at the embryo shield stage were put into every well, set up 3 replicates at each concentration, every culture dish as a replicate, and incubating in a multifunctional incubator at 26±1° C. with a photoperiod of 14 h (light): 10 h (dark);
E. Single toxicity of pesticides to zebrafish larvae and Japanese medaka: Design the toxicity test of pesticides to zebrafish and Japanese medaka according to the method of the OECD guidelines, based on the preliminary test to determine the effective concentration range of the pesticide, diluting the stock solution with standard dilution water to 5-7 concentrations with a geometrical ratio, using a 24-well cell culture dish as the poisoning device, the volume of each well is 3 mL, adding 2 mL of test solution to each well and a larva that has developed normally and just entered the migratory period through microscopy during the experiment, no feeding during the test, each concentration is set up in triplicate, every culture dish as a replicate, the zebrafish test temperature is 26±1° C., the test temperature is 25±1° C., the photoperiod is 14 h (light): 10 h (dark), replacing the test solution every 24 h, observing and counting the number of dead larvae every 24 h, and calculating LC₅₀ values and their 95% confidence limits by the probit analysis method when exposed 24 h, 48 h, 72 h and 96 h;
F. Combined toxicity test: The toxicity test is performed on zebrafish larvae and Japanese medaka larvae, the test procedure is as follows:
a. Zebrafish larvae: The LC₅₀ value of zebrafish larvae with a single pesticide for 96 h is a toxic unit, and 5-7 different concentrations with a geometrical ratio, test method and calculation of LC₅₀ value of each exposure time are the same as 1.4.2;
b. Japanese medaka: A single pesticide is used to measure the LC₅₀ value of Japanese medaka for 96 hours, mixing chlorpyrifos and butachlor to form binary mixed systems with different ratios of 1:4, 2:3, 1:1, 3:2 and 4:1, according to the pre-experiment results, 5-7 different concentrations are set at equal logarithmic intervals to determine the combined toxicity of the mixed system to Japanese medaka, the method is the same as the determination of single toxicity, the total concentration of the binary mixture is the sum of the concentrations of the two components;
G. Combined toxicity evaluation method: Using the following formula to find the sum of biological toxicity 5: S=Am/Ai+Bm/Bi, wherein Am and Bat are the toxicities of each pesticide in the mixture, and Ai and Bi are the toxicities of A and B pesticides when acting alone; convert S into additive index AI, when S≤1, AI=(1/S)−1.0; when S>1, AI=1.0−S, and evaluating the compound effect of chemicals with AI, when −0.2<AI<0.25, that is, addition; when it is AI≥0.25, it is greater than the addition effect, that is, synergistic effect; when AI≤−0.2 is less than the additive effect, that is, antagonism;
H. Data processing: Calculating the LC₅₀ value of pesticides on larvae and their 95% confidence limits by probit analysis based on the number and time of dead fish larvae, and using 95% confidence limit of LC50 as the criterion to determine whether the toxicity difference of different pesticides is significant, LC₅₀≤0.1 mg a.i. L⁻¹, is hypertoxic; 0.1<LC₅₀≤1.0 mg a.i. L⁻¹, is high toxicity; 1.0<LC₅₀≤10.0 mg a.i. L⁻¹, is medium toxicity; LC₅₀>10.0 mg a.i. is low toxicity. The maximum allowable concentration of MPC employs 100 as the protection factor, the formula is: MPC=96 h-LC₅₀/100, to get the maximum allowable concentration of a poison.

2. The test method for the combined toxicity of chlorpyrifos and butachlor according to claim 1, wherein the test water temperature in step A is controlled at 25±1° C., and the light cycle is 14 h of light and 10 h of darkness.

3. The test method for the combined toxicity of chlorpyrifos and butachlor according to claim 1, wherein in step B, using a Lycra S8AP0 type apochromatic stereo microscope for observation and photographing.

4. The test method for the combined toxicity of chlorpyrifos and butachlor according to claim 1, wherein the larvae in the migratory period in step E refer to fish 120 h after fertilization of eggs.

5. The test method for the combined toxicity of chlorpyrifos and butachlor according to claim 1, wherein the mixing ratio in the step F is designed with reference to a more toxic agent.

6. The test method for the combined toxicity of chlorpyrifos and butachlor according to claim 1, wherein the MPC in the step 1 is the maximum allowable concentration.

7. The test method for the combined toxicity of chlorpyrifos and butachlor according to claim 1, wherein in step 1, the toxicity classification standard of pesticides for larvae is based on “Environmental Safety Evaluation Test Guidelines of Chemical Pesticides” formulated by the State Environmental Protection Administration of China in 1989.

8. The test method for the combined toxicity of chlorpyrifos and butachlor according to claim 1, wherein in the step D, the test solution needs to be replaced every 24 h, observing and microscopical observing the CK group and the exposed group every 24 h, recording the number of embryos with normal development and malformations, and calculating the number of embryos hatched and larvae malformations, the experiment lasts 96 hours.
